(19) United States
(12) Patent Application Publication
(10) Pub. No.: US 2024/0199642 A1
(43) Pub. Date: Jun. 20, 2024

(54) METHOD FOR OBTAINING RIFAPENTINE WITH A NEW CRYSTALLINE FORM

(71) Applicant: INTERQUIM S.A. DE C.V., Cuautitlán Izcalli, Estado de México (MX)

(72) Inventors: Aaeón RODRIGUEZ WELTON, Coacalco de Berrozabála (MX); Diego Armando PÉREZ ROJAS, Coacalco de Berriozábal (MX); Blanca Miriam SANTOYO TEPOLE, Ciudad de México (MX)

(21) Appl. No.: 18/555,776

(22) PCT Filed: Apr. 21, 2021
(86) PCT No.: PCT/MX2021/050020

§ 371 Date: Oct. 17, 2023

(51) Int. Cl. C07D 498/08

(52) U.S. Cl. CPC C07D 498/08

(57) ABSTRACT

A new crystalline form of rifapentine, the characterization thereof and a method for obtaining same is disclosed. The method for obtaining the new crystalline form includes the steps of adding a mixture of dimethylformamide, tert-butylamine and paraformaldehyde to rifamycin S in the presence of glacial acetic acid; adding paraformaldehyde and heating; subsequently adding 1-amino-4-cyclopentylpiperazine to dimethylformamide; adding ascorbic acid to water; and mixing, filtering and washing the product The yield of raw rifapentine is 87+5% with 99.00% purity.

### FIELD OF INVENTION

The invention relates to the preparation of a new crystalline form of rifapentine.

### BACKGROUND

The present invention relates to the preparation of a new pure crystalline form of rifapentine, the new pure crystalline form as such and the pharmaceutical formulations containing it. Rifapentine is a rifamycin that is characterized by having a greater inhibition potential against the Mycobacterium tuberculosis bacteria and by having a longer half-life than rifampicin.

Rifapentine is the generic name of an antibiotic described in U.S. Pat. No. 4,002,752, particularly useful in the treatment of acute lung infections, M. Tsukamura et al., Kekkaku (Tuberculosis, Japan), 1986, 61/12, (633-639); PHYSICAL EDUCATION. Varaldo et al., Antimicrobial Agents Chemother. (United States), 1985, 27/4, (615-618); Yi Luy col., Chin. J. Antib. (China), 1987, 12/5, (341-344) and L. B. Heifets et al., Am. Rev. Respir. Dis 1990; 141, 626-360, describe the bactericidal activity of rifapentine against Mycobacterium avium.

Rifapentine obtained according to the process described in U.S. Pat. No. 4,002,752 is generally a mixture of different solid forms. These different forms show different characteristics of stability, formulability and bioavailability. In general, they are solvates or mixtures of solvates, depending on the “crystallization” solvent or solvents. It is mentioned that a pure amorphous or crystalline form is obtained through the process, but it is not characterized.

In publication WO 90/00553, Jan. 25, 1990, processes for obtaining Rifapentine salts are described: hydrochloride or hydrobromide, which have 2 crystalline forms and one amorphous form.

### Rifapentine Hydrochloride:

Form I, it is obtained by crystallization of Methanol.
Form II, it is obtained by crystallization of Acetone and re-crystallization of Ethanol/Chloroform.
Amorphous form, it is obtained by crystallization of ethyl acetate and precipitation in chloroform/ethyl ether.

### Rifapentine Hydrobromide:

Form I, it is obtained by crystallization in Methanol.
Form II, it is obtained by crystallization of Acetone.
Amorphous form, it is obtained by precipitation in Chloroform/Ethyl ether, without diffraction data.

The Publication WO 92/00302; Jan. 9, 1992 (EP 0536 197; Jul. 26, 1995) provides methods for obtaining a pure crystal Form I of Rifapentine, from a mixture of crystalline forms (Form I and an ethanol solvate SII). The process consists of dissolving the mixture of different crystalline forms of Rifapentine in methylene chloride at 30° C., adding Ethanol, removing the methylene chloride, until a constant temperature of the mixture (75-80° C.). Subsequently, a thermal treatment is carried out capable of transforming the mixture of Form I and SII to a pure crystalline Form I.

In Grown, characterization and crystal structure analysis of rifapentine. Zhou, K., Li, J. and Zheng, D. S., Journal of Molecular Structure, 983, (2010), 27-31 and Crystal Growth, Structure and Mmphology of Rifapentine Methanol Solvate. Chinese Journal of Chemical Engineering, 20 (3) 602-607 (2012), a crystalline form of Rifapentine obtained from Methanol is described, where Rifapentine was dissolved in Methanol at 30° C. until saturation and then cooled at a rate of 1° C./d. The molecular configuration of Rifapentine and the atom numbering scheme are shown in FIG. 3.

FIG. 3 is a perspective view of the molecular structure of Rifapentine. The glass is packaged as a stack. In the compound molecules, the five-membered ring of furan and naphthalene are coplanar. At the same time, the six-membered ring of piperazine and the five-membered ring of cyclopentyl have a chair conformation. The structure of Rifapentine is Monoclinic. In the Diffractogram, sharp peaks are observed at diffraction angles (2°θ): 5.57°, 6.65°, 7.9°, 10.29°, 14.83°, 15.66°, 21.28° and 22.74°.

In Crystal modification of rifapentine using different solvents. Zhou, K, Li, J., Luo, J. and Dongsheng, Z. Front. Chem. Eng. China 2010, 4(1): 65-69, different types of Rifapentine crystal are obtained by crystallization with different solvents. It is mentioned that the Rifapentine sample used was provided by Leshan San Jiu-Long March Pharmaceuticals Co., Ltd., China. The solvents used were: Methanol, Ethanol, Chloroform and Acetic acid. The preparation process for Rifapentine crystals consists of dissolving Rifapentine in 30 mL of the selected solvent. The solution is stirred at 50° C. for 2 hours and it is cooled at 20° C. for 4 hours. It is filtered under vacuum and washed with distilled water. The final product is kept in a tightly closed container until use. In the Diffractogram, sharp peaks are observed at diffraction angles (2°θ): 5.57°, 6.65°, 7.9°, 10.29°, 14.83°, 15.66°, 21.28° and 22.74°, these are obtained from untreated and crystallized Rifapentine with Ethanol, Methanol, Chloroform and Acetic acid respectively. The comparison of the diffractogram presented in FIG. 3 and the data recorded therein clearly shows that there is a significant difference in the complete diffraction pattern or gap values between the treated and untreated rifapentine samples. The intensity of the peaks of untreated Rifapentine is lower than the modified crystals reported in the article. This is probably due to the high perfection of the crystal under crystallization conditions. Furthermore, it is observed that the crystalline structure changes with Acetic acid, especially in the interval of 2°θ between 17° to 20°. Likewise, it is observed that Rifapentine crystallized with methanol corresponds to the crystalline form reported in the article of Journal of Molecular Structure, 983, (2010), 27-31.

Two crystalline forms of Rifapentine are described in A Novel Inhalable Form of Rifapentine. Journal of Pharmaceutical Sciences, 103: 1411-1421, (2014). Thus, 4 mg/mL of Rifapentine is dissolved in a co-solvent system of Acetone and deionized water (1:1, v/v). Choosing the co-solvent system to achieve a reasonable concentration of Rifapentine using a relatively non-toxic solvent. The Acetone is removed by heating at 67°, obtaining an aqueous suspension of crystalline Rifapentine. The suspension was homogenized at 10,000 rpm for 2 minutes to obtain a small and uniform crystal size. This suspension was spray dried. For its part, amorphous Rifapentine was prepared by dissolving 4 mg/mL of Methanol and it was spray-dried. The X-ray diffractogram showed multiple strong intensity peaks and a “halo” pattern typical for crystalline (top) and amorphous (bottom) Rifapentine powder.

New, stable forms of rifapentine are still needed to meet stability requirements under both normal storage and formulation conditions in order to optimize the preparation, formulation, storage and administration steps in a precise, reliable and standardized manner. Furthermore, a stable and well-defined crystalline form of an active ingredient is often a precondition and even a guarantee of a reliable and precise bioavailability pattern that is not subject to relevant batch-to-batch variations.

Sometimes excessive hygroscopicity of an active ingredient can also represent a delicate problem in storage and formulation procedures. The crystalline form of rifapentine described is found to be non-hydroscopic under normal handling and storage conditions.

### BRIEF DESCRIPTION OF THE DRAWINGS

FIG. 1 is a diffraction pattern of the crystalline form of Rifapentine obtained according to the present invention.

FIG. 2 shows the synthesis scheme of Rifapentine according to the present invention.

FIG. 3 illustrates the molecular configuration of Rifapentine and the atom numbering scheme.

### DESCRIPTION OF THE INVENTION

An object of this invention therefore, is a pure crystalline form of rifapentine, which is obtained in pure form according to the process of the invention. With the “pure crystalline form” it is intended that the crystalline form described is a solid made up of rifapentine crystals.

This pure crystalline form demonstrates to be stable and non-hygroscopic under normal storage conditions and normal formulation manipulations. Stability throughout the formulation and storage process is important for reliable bioavailability of the administered dose.

Another object of the invention is the process of preparing the pure crystalline form of rifapentine.

The synthesis process of rifapentine, object of the present invention, consists of mixing Dimethylformamide, tert-Butylamine and paraformaldehyde; adding Rifamycin S and glacial Acetic acid at 40° C.±5; adding paraformaldehyde and heating at 62-68° C. for 2 hours. Adding a solution of 1-Amino-4-cyclopentyl piperazine in Dimethylformamide at a temperature of 68° C. for 16 hours. Cooling the reaction mixture at 50° C. Adding a solution of ascorbic acid in water (400 mL) and mixing at 55-60° C. Adding ethyl acetate and water, cooling at 5-20° C. Filtering and washing the product with a mixture of Ethanol/water (1:1). Squeeze and dry under vacuum at 60-65° C. The yield of crude Rifapentine is 87±5% with a purity of 99.00%.

The crude product is purified by dissolving the solid in a mixture of Ethanol and water (6:1) and heating under reflux for approximately 1 hour. The mixture is cooled at 15-20° C. and the pure Rifapentine is filtered, washed with a mixture of Ethanol/Water (1:1) and a second wash with 2 volumes of Water, it is squeezed and dried at 60-65° C. under vacuum.

A re-pulping process is carried out in Water, mixing pure Rifapentine with 8 volumes of water, under stirring, filtered and washed with 1 volume of water. It is squeezed and dried under vacuum at 60° C. for 16 hours. Pure Rifapentine is obtained with a yield of 73±3% and a purity of 99.50%.

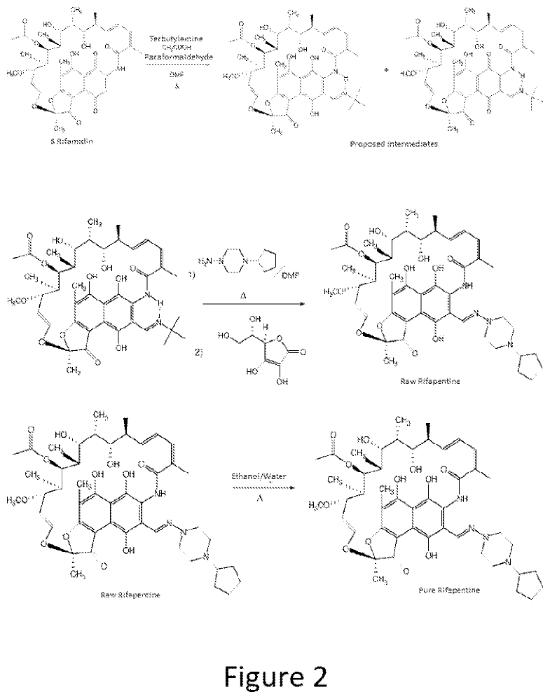

The synthesis scheme is shown in FIG. 2.

The pure crystalline form of Rifapentine can be formulated in various pharmaceutical dosage forms, such forms include oral solid forms such as capsules, tablets, pills, film-coated tablets, sugar-coated tablets, hard gelatin capsules, soft elastic capsules and the like. These forms may contain the crystalline form of pure rifapentine obtained according to the process in pharmaceutically effective doses in powder form mixed with the usual inert excipients such as binders, disintegrants, lubricants and preservatives. These additives are essentially the same as those that can be used in similar antibiotic formulations, for example rifampicin.

Other dosage forms of pharmaceutical products that can be used for oral administration or external applications include solutions, syrups, suspensions, emulsions and the like. Also in these cases, the method and ingredients normally used for the formulation of similar antibiotics, for example, rifampicin can be used successfully. Pharmaceutical dosage forms for parenteral administration are also encompassed by this particular aspect of the invention. These pharmaceutical dosage forms include preparations suitable for intramuscular or intravenous administration which may optionally be formulated as a dry powder for reconstitution before use.

The pure crystalline form of rifapentine can also be administered for use in pharmaceutical forms such as sprays, ointments and suppositories. The manufacture of pharmaceutical dosage forms containing the crystalline form of pure rifapentine can be carried out by commonly known procedures, see, for example: Remington's Pharmaceuticals Sciences, 17 Edition, (1985) Mack Publishing Co., East, Pennsylvania 18042.

The dosage forms above mentioned show good long-term stability under normal storage conditions.

### Example of Obtaining Rifapentine:

### Obtaining Raw Rifapentine.

Mixing 680 mL of Dimethylformamide, 60.4 mL of tert-Butylamine and 17.2 g paraformaldehyde, stirring for 5 minutes.

Next, 200 g of Rifamycin S are added and 65.6 mL of glacial acetic acid are added, slowly taking care that the temperature does not exceed 40° C.±5.

17.2 g of paraformaldehyde are added and heated to 62-68° C., maintaining this temperature for 2 hours.

Once the reaction time is over, 63.2 g of 1-Amino-4-cyclopentyl piperazine, previously dissolved in 140 mL of Dimethylformamide, are slowly added. Taking care that the temperature does not exceed 68° C., maintaining the temperature between 62-68° C. for 16 hours. Once the reaction is finished, it is cooled to 50° C.

A solution of ascorbic acid (25.3 g) in water (400 mL) is added, stirring for 20 minutes at 55-60° C. 300 mL of ethyl acetate are added, maintaining 55-60° C., and 800 mL of water are added, taking care that the temperature does not drop below 55° C.

Once the water has been added, it is cooled to 15-20° C., maintaining the temperature at 15-20° C. for 30 minutes.

The product is filtered and washed with 400 mL of Ethanol/water mixture (1:1); the product is squeezed for 60 minutes and dried under vacuum at 60-65° C. for 16 hours.

### Purification

6 volumes of Ethanol and water mixture (6:1) are added to the raw Rifapentine and it is heated at reflux for 60 minutes. Then it is cooled at 15-20° C., maintaining this temperature for 30 minutes. The pure Rifapentine is filtered, washed with 2 volumes of Ethanol/Water mixture (1:1); a second wash is carried out with 2 volumes of Water, it is squeezed for 60 minutes and it is dried at 60-65° C. under vacuum for 16 hours.

### Re-Pulp in Water

8 volumes of water are loaded into a reactor containing pure Rifapentine and it is stirred for 60 minutes at room temperature. It is filtered and washed with 1 volume of water; it is squeezed for 60 minutes and it is dried under vacuum at 60° C. for 16 hours.

Characterization of the Crystalline Form of Rifapentine. X-Ray Diffraction

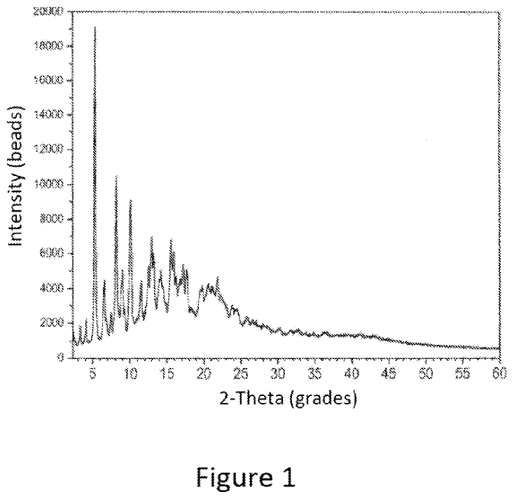

All X-ray powder diffraction measurements were carried out using a Briker D8 Discover equipment of Bragg Brentanol geometry Theta-Theta configuration, with a copper anode. The values obtained from the X-ray powder diffraction of Rifapentine according to the present invention are shown below in Table 1, the diffractogram of the crystalline form is shown in FIG. 1:

TABLE 1

| | | | | | Relative |
|---|---|---|---|---|---|
| | | | Net | Major | Intensity |
| Peak | Angle | d Value | Intensity | Intensity | (%) |
| 1 | 3.253602 | 27.13354 | 468.1783 | 1186.525 | 3.5 |
| 2 | 4.053458 | 21.78098 | 655.2691 | 1422.152 | 4.8 |
| 3 | 5.32029 | 16.59714 | 13549.94 | 14534.03 | 100.0 |
| 4 | 6.480815 | 13.62745 | 2097.838 | 3279.996 | 15.5 |
| 5 | 6.721023 | 13.14093 | 727.9749 | 1948.025 | 5.4 |
| 6 | 7.415725 | 11.91138 | 757.8583 | 2087.599 | 5.6 |
| 7 | 8.206759 | 10.76495 | 7026.619 | 8475.433 | 51.9 |
| 8 | 8.981197 | 9.838367 | 2812.366 | 4359.428 | 20.8 |
| 9 | 9.237077 | 9.566397 | 1052.06 | 2627.597 | 7.8 |
| 10 | 10.0463 | 8.797571 | 6685.828 | 8415.775 | 49.3 |
| 11 | 10.81314 | 8.17533 | 424.5566 | 2313.589 | 3.1 |
| 12 | 11.47483 | 7.705346 | 1949.113 | 3961.122 | 14.4 |
| 13 | 12.51409 | 7.067681 | 3018.303 | 5196.734 | 22.3 |
| 14 | 12.95888 | 6.826077 | 4509.406 | 6749.082 | 33.3 |
| 15 | 13.9619 | 6.70385 | 3043.244 | 5313.149 | 22.5 |
| 16 | 13.95179 | 6.342435 | 2049.521 | 4404.334 | 15.1 |
| 17 | 14.17191 | 6.244416 | 2198.406 | 4574.709 | 16.2 |
| 18 | 14.4022 | 6.145081 | 1548.671 | 3945.886 | 11.4 |
| 19 | 15.5531 | 5.69285 | 3983.746 | 6461.441 | 29.4 |
| 20 | 15.88097 | 5.576048 | 2981.098 | 5474.387 | 22.0 |
| 21 | 16.2422 | 5.452842 | 1976.644 | 4483.364 | 14.6 |
| 22 | 16.86293 | 5.253491 | 1917.18 | 4437.733 | 14.1 |
| 23 | 17.23041 | 5.142266 | 2310.659 | 4833.918 | 17.1 |
| 24 | 17.67462 | 5.014016 | 2204.283 | 4725.37 | 16.3 |
| 25 | 18.23749 | 4.860517 | 489.9084 | 2999.65 | 3.6 |
| 26 | 19.44481 | 4.561367 | 1318.754 | 3816.172 | 9.7 |
| 27 | 19.72785 | 4.496558 | 1417.902 | 3916.036 | 10.5 |
| 28 | 20.27315 | 4.376925 | 1182.523 | 3675.306 | 8.7 |
| 29 | 20.69687 | 4.288166 | 1607.148 | 4089.389 | 11.9 |
| 30 | 21.04304 | 4.218399 | 1629.373 | 4099.053 | 12.0 |
| 31 | 21.79808 | 4.073959 | 1806.693 | 4236.405 | 13.3 |
| 32 | 22.30871 | 3.98185 | 1153.567 | 3546.475 | 8.5 |
| 33 | 23.03471 | 3.857966 | 794.3196 | 3121.329 | 5.9 |
| 34 | 23.77201 | 3.739953 | 756.6824 | 3000.456 | 5.6 |
| 35 | 2433162 | 3.655189 | 602.9971 | 2772.62 | 4.5 |
| 36 | 25.80754 | 3.4494 | 317.5044 | 2337.964 | 2.3 |
| 37 | 26.4803 | 3.36327 | 179.5939 | 2153.73 | 1.3 |
| 38 | 26.94004 | 3.306909 | 216.9332 | 2152.548 | 1.6 |
| 39 | 28.05061 | 3.178455 | 227.892 | 2042.676 | 1.7 |
| 40 | 28.57614 | 3.121186 | 169.708 | 1915.33 | 1.3 |
| 41 | 30.10092 | 2.966461 | 195.0133 | 1741.518 | 1.4 |
| 42 | 31.48765 | 2.838908 | 160.8284 | 1588.574 | 1.2 |
| 43 | 31.77455 | 2.813927 | 142.7208 | 1563.021 | 1.1 |
| 44 | 32.72852 | 2.734052 | 259.3916 | 1637.036 | 1.9 |
| 45 | 33.5844 | 2.666307 | 122.08 | 1438.033 | 0.9 |
| 46 | 34.72667 | 2.581174 | 112.561 | 1397.725 | 0.8 |
| 47 | 36.29156 | 2.473382 | 193.5269 | 1472.665 | 1.4 |
| 48 | 42.94948 | 2.104121 | 75.30029 | 1247.114 | 0.6 |

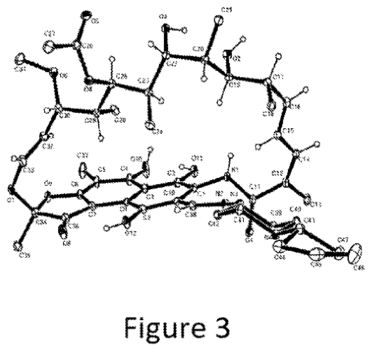

FIGS. 2 to 6 show the comparison of the X-ray powder diffraction pattern of the crystalline form of Rifapentine of the present invention with the reflections of the Crystalline Forms reported in WO 90/00553, WO 92/00302, Zhou, Li & Zheng, J. Molecular Structure (2010) and Zhou, Li & Zheng, Chem. Eng. China (2010), where it is shown that the crystalline form of Rifapentine of the present invention Form I does not correspond to any of the diffraction patterns reported in the state of the art. It should be noted that the “monoclinic” crystalline form reported in the publications of Zhou, K. et al., shows a variation in the position of 2-Theta greater than 0.2 degree, compared to the diffraction pattern of the crystalline form of Rifapentine according to the present invention.

1. A pure crystalline form of rifapentine having an X-ray powder diffraction pattern with the values shown in Table 1.

2. The pure crystalline form of rifapentine having the X-ray powder diffractogram according to FIG. 1.

3. A process for the preparation of the pure crystalline form of rifapentine comprising: mixing Dimethylformamide, tert-Butylamine and paraformaldehyde; adding Rifamycin S and glacial Acetic acid at 40° C.±5; adding paraformaldehyde and heating at 62-68° C. for 2 hours; adding a solution of 1-Amino-4-cyclopentyl piperazine in Dimethylformamide at a temperature of 68° C. for 16 hours; cooling the reaction mixture at 50° C.; adding a solution of ascorbic acid in water (400 mL) and mixing at 55-60° C.; adding ethyl acetate and water, cooling at 5-20° C.; filtering and washing the product with a mixture of Ethanol/water (1:1); squeezing and dry under vacuum at 60-65° C.; purify crude rifapentine and carry out re-pulping of the product.

4. The process according to claim 3, characterized by the crude product is purified by dissolving the solid in a mixture of Ethanol and water (6:1) and heating at reflux for approximately 1 hour; cooling the mixture at 15-20° C. and filtering the pure Rifapentine; washing with a mixture of Ethanol/Water (1:1) and a second wash with 2 volumes of Water, squeeze and dry at 60-65° C. under vacuum.

5. The process according to claim 3, characterized by the re-pulping stage in water is carried out by mixing the pure Rifapentine with 8 volumes of water, under stirring, filtering and washing with 1 volume of water; squeeze and dry under vacuum at 60° C. for 16 hours.

6. The process according to claim 3, characterized by the yield of crude Rifapentine is 87±5% with a purity of 99.00%.

7. The process according to claim 3, characterized by pure Rifapentine is obtained with a yield of 73±3% and a purity of 99.50%.

8. A pharmaceutical composition having the pure crystalline form according to claim 1 as an active ingredient.
